(19) United States
(12) Patent Application Publication
(10) Pub. No.: US 2006/0287351 A1
(43) Pub. Date: Dec. 21, 2006

(54) Antibiotic cycloalkyltetrahydroquinoline derivatives

(76) Inventors: Richard F. Labaudiniere, Sherborn, MA (US); Yibin Xiang, Acton, MA (US); Ravi K. Jalluri, Avondale, PA (US); Anthony C. Arvanites, New Bedford, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/568,252

(22) PCT Filed: Aug. 11, 2004
(86) PCT No.: PCT/US04/25937

§ 371 Date: Aug. 2, 2006

Related U.S. Application Data
(60) Provisional application No. 60/494,669, filed on Aug. 13, 2003.

(51) Int. Cl. A61K 31/473

(52) U.S. Cl. 514/290

(57) ABSTRACT

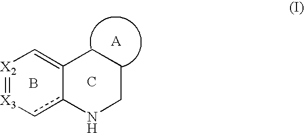

A method of treating a subject for a bacterial infection includes administering to a subject in need of treatment for a bacterial infection an effective amount of a compound represented by structural formula (I), or a pharmaceutically acceptable salt, solvate, or hydrate thereof. The variables in structural formula (I) are described herein.

### RELATED APPLICATIONS

This application claims priority to and is a continuation of U.S. Application No. 60/494,669, filed on Aug. 13, 2003, the entire teachings of which are incorporated herein by reference.

### BACKGROUND OF THE INVENTION

In the last century, antibiotics were developed that led to significant reductions in mortality. Unfortunately, widespread use has led to the rise of antibiotic resistant bacteria, e.g., methicillin resistant Staphyloccocus aureus (MRSA), vancomycin resistant enterococci (VRE), and penicillin-resistant Streptococcus pneumoniae (PRSP). Some bacteria are resistant to a range of antibiotics, e.g., strains of Mycobacterium tuberculosis resist isoniazid, rifampin, ethambutol, streptomycin, ethionamide, kanamycin, and rifabutin. In addition to resistance, global travel has spread relatively unknown bacteria from isolated areas to new populations. Furthermore, there is the threat of bacteria as biological weapons. These bacteria may not be easily treated with existing antibiotics.

Infectious bacteria employ the peptidoglycan biosynthesis pathway, and in particular, depend on MurA (phosphoenolpyruvate:UDP-N-acetyl-D-glucosamine 1-carboxyvinyltransferase, EC 2.1.5.7), to catalyze the transformation of uridine diphosphate-N-acetyl-D-glucosamine and phosphoenolpyruvate into uridine diphosphate-N-acetyl-3-O-(1-carboxyvinyl)-D-glucosamine: 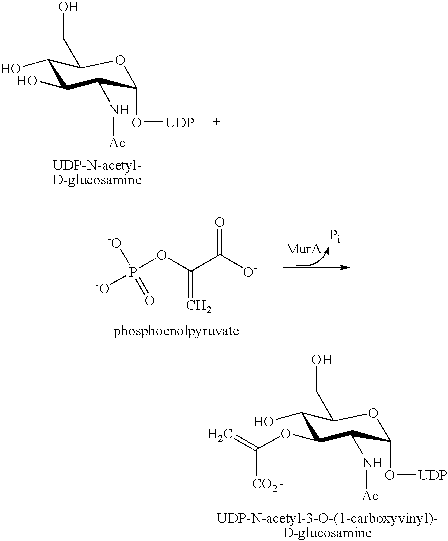
MurA is conserved across both Gram positive and Gram negative bacteria, but is not present in mammalian systems, and is thus a desirable and selective target for new medications.

Therefore, there is a need for new medications that target MurA, whereby infections from bacteria dependent on MurA can be treated.

### SUMMARY OF THE INVENTION

It has now been found that certain cycloalkyltetrahydroquinoline derivatives strongly inhibit MurA, as shown in Example 3. A number of the disclosed inhibitors are found to have antibiotic activity against bacteria, including drug-resistant bacteria, as shown in Example 4. Furthermore, many of the disclosed MurA inhibitors have low cytotoxicity, as shown in Example 5. Based on these discoveries, compounds that are MurA inhibitors, methods of treatment with the disclosed MurA inhibitors, and pharmaceutical compositions comprising the disclosed MurA inhibitors, and methods for screening for MurA inhibitors are provided herein.

A method of treating a subject for a bacterial infection includes administering to a subject in need of treatment for a bacterial infection an effective amount of a compound represented by structural formula I: 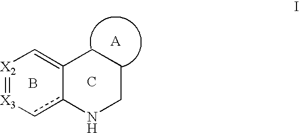
or a pharmaceutically acceptable salt, solvate, or hydrate thereof.

Ring A is a 5 or 6 membered cycloalkyl or cycloalkenyl group, optionally substituted with halogen or optionally halogenated C1-C3 alkyl or alkoxy.

X2 and X3 are each carbon, or one is nitrogen and the other is carbon.

Rings B and C are optionally and independently substituted at any substitutable ring carbon, provided that one or two substitutable ring carbons in Rings B and C are substituted with an acidic group.

In another embodiment, the acidic group is selected from —(CO)OH, —(CS)OH, —(SO)OH, —SO₃H, —OSO₃H, —P(OR^{a})(OH), —(PO)(OR^{a})(OH), —O(PO)(OR^{a})(OH), or —B(OR^{a})(OH), wherein R^{a} is —H or optionally substituted aryl, aralkyl, heteroaryl, heteroaralkyl, or C1 to C4 alkyl. Typically, the acidic group is —(CO)OH, —(CS)OH, —(SO)OH, —SO₃H, —OSO₃H, or preferably, —(CO)OH.

Another embodiment is a method of identifying a MurA inhibitor, including contacting MurA with phosphoenolpyruvate and a test compound, under conditions suitable for reaction between the MurA enzyme and the substrate phosphoenolpyruvate, and determining a reaction rate between the phosphoenolpyruvate and MurA. The test compound is identified as a MurA inhibitor when the rate of reaction in the presence of the test compound is less than a reaction rate in the absence of the test compound. More preferably, the method includes conducting the reaction in the presence of MurB and uridine 5′-diphospho-N-acetylglucosamine. In a preferred embodiment, the method of identifying compounds as MurA inhibitors is combined with one or more assays for antibiotic activity. Such assays are well known in the art, and can include, for example, contacting bacteria of interest with a test compound under conditions otherwise suitable for bacterial growth, and determining if the test compound has antibacterial activity.

The invention is useful for treating (therapeutically or prophylactically) bacterial infections, particularly infections caused by bacteria that depend on the peptidoglycan biosynthesis pathway, and more particularly, infections caused by bacteria that express the MurA enzyme. Furthermore, it can be useful against bacteria that have developed antibiotic resistance, especially multiple drug resistant strains, because it is believed to act through a: different mechanism than existing, widely used antibiotics.

### DETAILED DESCRIPTION OF THE INVENTION

The invention is generally related to methods, compounds, and pharmaceutical compositions for treating and preventing bacterial infections. In particular, the invention relates to substituted cycloalkyltetrahydroquinoline derivatives that are MurA inhibitors.

In preferred embodiments, the MurA inhibitor of the method is represented by one of structural formulas I-a to I-c or I-a″:

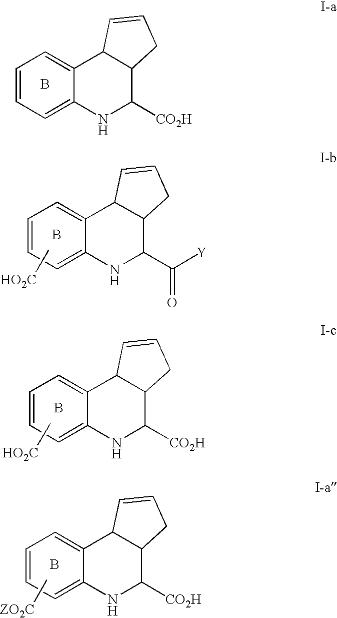

In I-a″, Z is —H or a C1 to C4 alkyl group.

In I-b, Y is optionally substituted C1 to C4 alkyl, C1 to C4 alkoxy, phenyl, pyridyl, or —NR^{j}₂, wherein each R^{j} is independently —H, C1 to C4 alkyl, aryl, or aralkyl, or NR^{j}₂ is a nonaromatic heterocycle.

In structural formulas I-a to I-c and I-a″, Ring B is optionally substituted at any substitutable ring carbon.

In more preferred embodiments, the MurA inhibitor is represented by one of structural formulas I-a′ to I-c′:

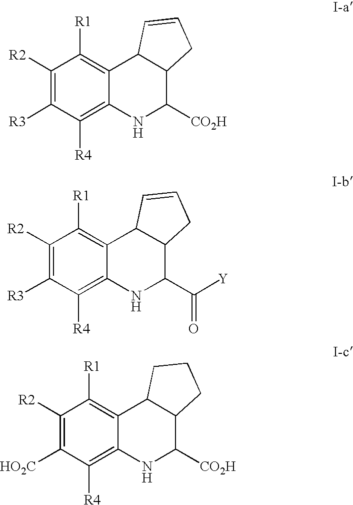

The variables R1, R2, R3 and R4 are independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, nonaromatic heterocycle, C1 to C4 alkyl, C1 to C4 alkoxy, C1 to C4 hydroxy alkyl, or C2 to C6 alkoxyalkyl; provided that, for I-b′, at least one of R1 to R4 is an acidic group, e.g., —CO₂H. In a preferred embodiment of I-a′, at least one of R1 to R4 is —CO₂H, and the remainder of R1 to R4 are as described above.

Each R^{b} and R^{d} is independently —H or optionally substituted aryl, aralkyl, heteroaryl, heteroaralkyl, or C1 to C4 alkyl, and each R^{c} is independently —H or optionally substituted C1 to C4 alkyl, aryl, or aralkyl, or NR^{c}₂ is an optionally substituted nonaromatic heterocycle. More typically, each R^{b}, R^{c}, and R^{d} is independently —H, or optionally substituted C1 to C4 alkyl or phenyl, or each NR^{c}₂ is an optionally substituted morpholinyl, piperidyl, or piperazyl. Preferably, each R^{b}, R^{c} and R^{d} is independently —H or C1 to C4 alkyl; or NR^{c}₂ is a nonaromatic heterocycle.

In preferred embodiments of I-a′, I-b′, and I-c′, at least two of R1 to R4 are —H, or more typically, at least two of R1, R2, and R4 are —H. More typically two, and preferably three of R1 to R4 are —H, or two of R1, R2, and R4 are —H.

More preferably for I-a′, one or two of R1 to R4 are each independently halogen —(CO)R^{b}, —(CO)OR^{b}, —(CO)NR^{c}₂, —NR^{c}₂, —NR^{d}(CO)R^{b}, NR^{d}(CO)OR^{b}, —NR^{d}(CO)NR^{c}₂, —NR^{d}(CO)PhNR^{d}(CO)R^{b}, or optionally substituted phenyl, benzyl, pyridyl, methylpyridyl, or optionally halogenated C1 to C4 alkyl or C1 to C4 alkoxy. In another preferable embodiment of I-a′, R1, R2, R3, and R4 are independently —H, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, or nonaromatic heterocycle. More preferably, one or two of R1 to R4 are each independently —(CO)R^{b}, —(CO)OR^{b}, —(CO)NR^{c}₂, —NR^{c}₂, —NR^{d}(CO)R^{b}, —NR^{d}(CO)OR^{b}, —NR^{d}(CO)NR^{c}₂, —NR^{d}(CO)PhNR^{d}(CO)R^{b}, or optionally substituted phenyl, benzyl, pyridyl, or methylpyridyl;

More preferably, for I-b′, R1 to R4 are as described in the preceding paragraph, provided that at least one of R1 to R4 is an acidic group, e.g., —CO₂H.

More preferably for I-c′, R1, R2, and R4 are independently —H, —F, —Cl, —Br, —NO₂, —CN, —(CO)R^{b}, —(CO)NR^{c}₂, —NR^{c}_{2,} NR^{d}(CO)R^{b}, —NR^{d}(CO)OR^{b}, —NR^{d}(CO)NR^{c}₂, —SO₂NR^{c}₂, —NR^{d}SO₂R^{b}, or optionally halogenated C1 to C4 hydroxy alkyl, C1 to C4 alkyl, or C1 to C4 alkoxy.

In other preferred embodiments of I-a′ and I-b′, at least one of R1 to R4 is —(CO)OR^{b}, e.g., —CO₂H or a C1-C4 carboxylic ester thereof. More typically, at least one of R1 to R4 is —CO₂H, or preferably, one of R1 to R3 is —CO₂H.

Specific examples of MurA inhibitors of the present invention are the compounds in Table 1.

Also included in the present invention are pharmaceutical compositions comprising the disclosed MurA inhibitors, (e.g., I-b, I-c, I-a′ to I-c′, and I-a″). The present invention also includes novel MurA inhibitors disclosed herein (e.g., I-b, I-c, I-a′ to I-c′, and I-a″), or pharmaceutically acceptable, salts, solvates or hydrates thereof.

A “subject” includes mammals, e.g., humans, companion animals (e.g., dogs, cats, birds, aquarium fish, reptiles, and the like), farm animals (e.g., cows, sheep, pigs, horses, fowl, farm-raised fish and the like) and laboratory animals (e.g., rats, mice, guinea pigs, birds, aquarium fish, reptiles, and the like). Alternatively, the subject is a warm-blooded animal. More preferably, the subject is a mammal. Most preferably, the subject is human.

A subject in need of treatment has a bacterial infection (or has been exposed to an infectious environment where bacteria are present, e.g., in a hospital) the symptoms of which may be alleviated by administering an effective amount of the disclosed MurA inhibitors. For example, a subject in need of treatment can have an infection for which the disclosed MurA inhibitors can be administered as a treatment. In another example, a subject in need of treatment can have an open wound or burn injury, or can have a compromised immune system, for which the disclosed MurA inhibitors can be administered as a prophylactic. Thus, a subject can be treated therapeutically or prophylactically. More preferably, a subject is treated therapeutically.

Typically, the subject is treated for a bacterial infection caused by a bacteria of a genus selected from Allochromatium, Acinetobacter, Bacillus, Campylobacter, Chlamydia, Chlamydophila, Clostridium, Citrobacter, Escherichia, Enterobacter, Enterococcus, Francisella, Haemophilus, Helicobacter, Klebsiella, Listeria, Moraxella, Mycobacterium, Neisseria, Proteus, Pseudomonas, Salmonella, Serratia, Shigella, Stenotrophomonas, Staphyloccocus, Streptococcus, Synechococcus, Vibrio, and Yersina.

More preferably, the subject is treated for a bacterial infection from Allochromatium vinosum, Acinetobacter baumanii, Bacillus anthracis, Campylobacter jejuni, Chlamydia trachomatis, Chlamydia pneumoniae, Clostridium spp., Citrobacter spp., Escherichia coli, Enterobacter spp., Enterococcus faecalis., Enterococcus faecium, Francisella tularensis, Haemophilus influenzae, Helicobacter pylori, Klebsiella spp., Listeria monocytogenes, Moraxella catharralis, Mycobacterium tuberculosis, Neisseria meningitidis, Neisseria gonorrhoeae, Proteus mirabilis, Proteus vulgaris, Pseudomonas aeruginosa, Salmonella spp., Serratia spp., Shigella spp., Stenotrophomonas maltophilia, Staphyloccocus aureus, Staphyloccocus epidermidis, Streptococcus pneunmoniae, Streptococcus pyogenes, Streptococcus agalactiae, Yersina pestis, and Yersina enterocolitica, and the like.

Preferably, the subject is treated for a bacterial infection caused by a bacterium that expresses a peptidoglycan biosynthesis pathway, and in particular, expresses the enzyme encoded by the MurA/MurZ gene. Numerous studies have demonstrated that the MurA gene and its paralog MurZ are conserved across a range of Gram positive and Gram negative bacteria; see, for example, Schonbrunn E, Eschenburg S, Krekel F, Luger K, Amrhein N. (2000) Biochemistry. 2000 Mar. 7;39(9):2164-73; Baum E Z, Montenegro D A, Licata L, Turchi I, Webb G C, Foleno B D, Bush K. (2001) Antimicrob Agents Chemother. 2001 November;45(11):3182-8; Kim D H, Lees W J, Kempsell K E, Lane W S, Duncan K, Walsh C T. (1996) Biochemistry. 1996 Apr. 16;35(15):4923-8; and Skarzynski T, Mistry A, Wonacott A, Hutchinson S E, Kelly V A, Duncan K. (1996) Structure. 1996 Dec. 15;4(12):1465-74. The entire teachings of these documents are incorporated herein by reference.

As used herein, the term MurA, referring to the gene or the enzyme thereby encoded, encompasses both MurA and its paralog MurZ. The enzymes are given various names in the art, including, for example: MurA transferase; MurZ transferase; UDP-N-acetylglucosamine 1-carboxyvinyl-transferase; UDP-N-acetylglucosamine enoylpyruvyl transferase; UDP-N-acetyl glucosamine enolpyruvyltransferase; enoylpyruvate transferase; phosphoenolpyruvate-UDP-acetylglucosamine-3-enolpyruvyltransferase; phosphoenolpyruvate:UDP-2-acetamido-2-deoxy-D-glucose 2-enoyl-1-carboxyethyltransferase; phosphoenolpyruvate:uridine diphosphate N-acetyl glucosamine enolpyruvyltransferase; phosphoenolpyruvate:uridine-5′-diphospho-N-acetyl-2-amino-2-deoxyglucose 3-enolpyruvyltransferase; phosphopyruvate-uridine diphosphoacetylglucosamine pyruvatetransferase; pyruvate-UDP-acetyl glucosamine transferase; pyruvate-uridine diphospho-N-acetyl glucosamine transferase; pyruvate-uridine diphospho-N-acetyl-glucosamine transferase; or pyruvic-uridine diphospho-N-acetylglucosaminyltransferase.

As used herein, the term MurB, referring to the gene or the enzyme thereby encoded, is given various names in the art, including, for example: UDP-N -acetylmuramate dehydrogenase, MurB reductase; UDP-N-acetylenol pyruvoyl glucosamine reductase; UDP-N-acetylglucosamine-enoylpyruvate reductase; UDP -GlcNAc-enoylpyruvate reductase; uridine diphosphoacetylpyruvoylglucosamine reductase; uridine diphospho-N-acetylglucosamine-enolpyruvate reductase; uridine -5′-diphospho-N-acetyl-2-amino-2-deoxy-3-O-lactylglucose:NADP-oxidoreductase

The systematic name typically given for MurA/MurZ is phosphoenolpyruvate:UDP-N-acetyl-D-glucosamine 1-carboxyvinyltransferase, and the IUBMB systematic classification is EC 2.5.1.7. The systematic name typically given for MurB is UDP-N-acetylmuramate:NADP+oxidoreductase, and the IUBMB systematic classification is EC 1.1.1.158. See International Union of Biochemistry and Molecular Biology online at www.chem.qmul.ac.uk/iubmb/.

In other embodiments, bacterial growth can be retarded, modulated, or prevented by employing an effective amount of the disclosed MurA inhibitors. Numerous bacteria can express the MurA enzyme. Bacteria that express MurA can include, for example, actinobacteria, bacteroids, chlamydia, cyanobacteria; firmicutes, e.g., bacillales, clostridia, and lactobacillales; fusobacteria; green sulfur bacteria; hyperthermophilic bacteria; proteobacteria, e.g., alpha, beta, delta, epsilon, and gamma; radioresistant bacteria; and spirochetes.

For example, actinobacteria can include, Bifidobacterium longum, Corynebacterium efficiens, Corynebacterium glutamicum, Mycobacterium bovis, Mycobacterium leprae, Mycobacterium tuberculosis (e.g., CDC1551 and H37Rv (lab strain)), Streptomyces coelicolor, Tropheryma whipplei (e.g., Twist, TW08/27); and the like.

Examples of bacteroids include Bacteroides thetaiotaomicron and the like.

Chlamydia can include, e.g., Chlamydophila caviae, Chlamydia muridarum, Chlamydophila pneumoniae (e.g., AR39, J138, CWL029, Chlamydia trachomatis, and the like.

Examples of cyanobacteria can include Anabaena sp. PCC7120 (Nostoc sp. PCC7120), Synechocystis sp. PCC6803, Thermosynechococcus elongates, and the like.

Firmicutes, e.g., bacillales can include Bacillus cereus, Bacillus halodurans, Bacillus subtilis, Listeria innocua, Listeria monocytogenes, Oceanobacillus iheyensis, Staphylococcus aureus (e.g., MW2, N315, and Mu50), Staphylococcus epidermidis, and the like.

Firmicutes, e.g., clostridia, can include Clostridium acetobutylicum, Clostridium perfringens, Clostridium tetani, Thermoanaerobacter tengcongensis, and the like.

Firmicutes, e.g., lactobacillales, can include Enterococcus faecalis, Lactococcus lactis, Lactobacillus plantarum, Streptococcus agalactiae (e.g., 2603 and NEM316), Streptococcus mutans, Streptococcus pyogenes (e.g., MGAS315 (serotype M3), SF370 (serotype M1), SSI-1 (serotype M3), and MGAS8232 (serotype M18)), Streptococcus pneumoniae (e.g., TIGR4 and R6), and the like.

Fusobacteria can include Fusobacterium nucleatum, and the like.

Green sulfur bacteria can include Chlorobium tepidum, and the like

Hyperthermophilic bacteria can include Aquifex aeolicus, Thermotoga maritime, and the like.

Examples of alpha proteobacteria can include Agrobacterium tumefaciens C58 (Cereon), Bradyrhizobium japonicum, Brucella melitensis, Brucella suis, Caulobacter crescentus, Mesorhizobium loti, Rickettsia conorii, Rickettsia prowazekii, Sinorhizobium meliloti, and the like.

Examples of beta proteobacteria can include Nitrosomonas europaea, Neisseria meningitidis (e.g., Z2491 (serogroup A) and MC58 (setogroup B), Ralstonia solanacearum, and the like.

Examples of delta/epsilon proteobacteria can include Campylobacter jejuni, Helicobacter pylori (e.g., J99 and 26695), and the like.

Examples of gamma proteobacteria can include Buchnera aphidicola (e.g., Baizongia pistaciae), Buchnera aphidicola (e.g., Schizaphis graminum), Buchnera sp. APS (e.g., Acyrthosiphon pisum), Coxiella burnetii, Escherichia coli (e.g., CFT073, O157 EDL933, K-12 W3110, K-12 MG1655, and O157 Sakai), Haemophilus influenzae, Pseudomonas aeruginosa, Pasteurella multocida, Pseudomonas putida, Pseudomonas syringae pv., Shigella flexneri 301 (serotype 2a), Shewanella oneidensis, Salmonella typhimurium, Salmonella typhi (e.g., Ty2, CT18), Vibrio cholerae, Vibrio parahaemolyticus, Vibrio vulnificus, Wigglesworthia brevipalpis, Xanthomonas axonopodis, Xanthomonas campestris, Xylella fastidiosa (e.g., 9a5c and Temecula1), Yersinia pestis (e.g., CO92 and KIM), and the like.

Radioresistant bacteria can include Deinococcus radiodurans, and the like

Spirochetes can include Borrelia burgdorferi, Leptospira interrogans, Treponema pallidum, and the like.

In one embodiment, a subject is also concurrently treated for a fungal infection, for example, a fungal infection caused by a pathogenic dermatophyte, e.g., a species of the genera Trichophyton, Tinea, Microsporum, Epidermophyton and the like; or a pathogenic filamentous fungus, e.g., a species of genera such as Aspergillus, Histoplasma, Cryptococcus, Microsporum, and the like; or a pathogenic non-filamentous fungus, e.g., a yeast, for example, a species of the genera Candida, Malassezia, Trichosporon, Rhodotorula, Torulopsis, Blastomyces, Paracoccidioides, Coccidioides, and the like. Preferably, the subject is concurrently treated for a fungal infection resulting from a species of the genera Aspergillus or Trichophyton. Species of Trichophyton include, for example, T. mentagrophytes, T. rubrum, T. schoenleinii, T. tonsurans, T. verrucosum, and T. violaceum. Species of Aspergillus include, for example, A. fumigatus, A. flavus, A. niger, A. amstelodami A. candidus, A. carneus, A. nidulans, A oryzae, A. restrictus, A. sydowi, A. terreus, A. ustus, A. versicolor, A. caesiellus, A. clavatus, A. avenaceus, and A. deflectus. More preferably, the subject is concurrently treated therapeutically for a fungal infection caused by a species of the genus Aspergillus selected from A. fumigatus, A. flavus, A. niger, A. amstelodami, A. candidus, A. carneus, A. nidulans, A oryzae, A. restrictus, A. sydowi, A. terreus, A. ustus, A. versicolor, A. caesiellus, A. clavatus, A. avenaceus, and A. deflectus. Even more preferably the subject is concurrently treated therapeutically for a fungal infection caused by Aspergillus fumigatus or Aspergillus niger, and most preferably, Aspergillus fumigatus.

An “effective amount” of a compound of the disclosed invention is the quantity which, when administered to a subject in need of treatment, improves the prognosis of the subject, e.g., delays the onset of and/or reduces the severity of one or more of the subject's symptoms associated with a bacterial infection. The amount of the disclosed compound to be administered to a subject will depend on the particular disease, the mode of administration, co-administered compounds, if any, and the characteristics of the subject, such as general health, other diseases, age, sex, genotype, body weight and tolerance to drugs. The skilled artisan will be able to determine appropriate dosages depending on these and other factors. Effective amounts of the disclosed compounds typically range between about 0.01 mg/kg per day and about 100 mg/kg per day, and preferably between 0.1 mg/kg per day and about 10 mg/kg/day. Techniques for administration of the disclosed compounds of the invention can be found in Remington: the Science and Practice of Pharmacy, 19^{th} edition, Mack Publishing Co., Easton, Pa. (1995), the entire teachings of which are incorporated herein by reference.

A “pharmaceutically acceptable salt” of the disclosed compound is a product of the disclosed compound that contains an ionic bond, and is typically produced by reacting the disclosed compound with either an acid or a base, suitable for administering to a subject.

For example, an acid salt of a compound containing an amine or other basic group can be obtained by reacting the compound with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid.

Salts of compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N, N′-dibenzylethylenediamine, 2-hydroxyethylamine, bis-(2-hydroxyethyl)amine, tri-(2-hydroxyethyl)amine, procaine, dibenzylpiperidine, N-benzyl-β-phenethylamine, dehydroabietylamine, N,N′-bisdehydroabietylamine, glucamine, N-methylglucamine, collidine, quinine, quinoline, and basic amino acid such as lysine and arginine.

Certain compounds and their salts may also exist in the form of solvates, for example hydrates, and the present invention includes each solvate and mixtures thereof.

As used herein, a “pharmaceutical composition” is a formulation containing the disclosed compounds in a form suitable for administration to a subject. The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of active ingredient (i.e., a formulation of the disclosed compound or salts thereof) in a unit dose of composition is an effective amount and may be varied according to the particular treatment involved. It may be appreciated that it may be necessary to make routine variations to the dosage depending on the age and condition of the patient. The dosage will also depend on the route of administration. A variety of routes are contemplated, including topical, oral, pulmonary, rectal, vaginal, parenternal, transdermal, subcutaneous, intravenous, intramuscular, intraperitoneal and intranasal.

The compounds described herein, and the pharmaceutically acceptable salts thereof can be used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein. Techniques for formulation and administration of the disclosed compounds of the invention can be found in Remington: the Science and Practice of Pharmacy, above.

For oral administration, the disclosed compounds or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like.

For parental administration of the disclosed compounds, or salts, solvates, or hydrates thereof, can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations previously described, the compounds may also be formulated as a depot preparation. Suitable formulations of this type include biocompatible and biodegradable polymeric hydrogel formulations using crosslinked or water insoluble polysaccharide formulations, polymerizable polyethylene oxide formulations, impregnated membranes, and the like. Such long acting formulations may be administered by implantation or transcutaneous delivery (for example subcutaneously or intramuscularly), intramuscular injection or a transdermal patch. Preferably, they are implanted in, or applied to, the microenvironment of an affected organ or tissue, for example, a membrane impregnated with the disclosed compound can be applied to an open wound or burn injury. Thus, for example, the compounds may be formulated with suitable polymeric or hydrophobic materials, for example, as an emulsion in an acceptable oil, or ion exchange resins, or as sparingly soluble derivatives, for example, as a sparingly soluble salt.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, a cream formulation can be administer to a wound site, or a bandage may be impregnated with a formulation, and the like.

For rectal administration, suitable pharmaceutical compositions are, for example, topical preparations, suppositories or enemas.

For vaginal administration, suitable pharmaceutical compositions are, for example, topical preparations, pessaries, tampons, creams, gels, pastes, foams or sprays.

In addition, the compounds may also be formulated to deliver the active agent by pulmonary administration, e.g., administration of an aerosol formulation containing the active agent from, for example, a manual pump spray, nebulizer or pressurized metered-dose inhaler. Suitable formulations of this type can also include other agents, such as antistatic agents, to maintain the disclosed compounds as effective aerosols.

The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

For nasal administration, either a solid or a liquid carrier can be used. The solid carrier includes a coarse powder having particle size in the range of, for example, from about 20 to about 500 microns and such formulation is administered by rapid inhalation through the nasal passages. Where the liquid carrier is used, the formulation may be administered as a nasal spray or drops and may include oil or aqueous solutions of the active ingredients.

In addition to the formulations described above, a formulation can optionally include, or be co-administered with one or more additional drugs, e.g., other antibiotics, anti-inflammatories, antifungals, antivirals, immunomodulators, antiprotozoals, steroids, decongestants, bronchodialators, and the like. For example, the disclosed compound can be co-administered with drugs such as such as ibuprofen, prednisone (corticosteroid) pentoxifylline, Amphotericin B, Fluconazole, Ketoconazol, Itraconazol, penicillin, ampicillin, amoxicillin, and the like. The formulation may also contain preserving agents, solubilizing agents, chemical buffers, surfactants, emulsifiers, colorants, odorants and sweeteners.

The term “aryl” group, (e.g., the aryl groups represented by R1 to R4) refers to carbocyclic aromatic groups such as phenyl, naphthyl, tetrahydronaphthyl, anthracyl, and the like. The term “heteroaryl” group (e.g., the heteroaromatic groups represented by R1 to R4) refers to heteroaromatic groups, for example imidazolyl, isoimidazolyl, thienyl, furanyl, pyridyl, pyrimidyl, pyranyl, pyrazolyl, pyrrolyl, pyrazinyl, thiazolyl, isothiazolyl, oxazolyl, isooxazolyl, 1,2,3-triazolyl, 1,2,4-triazolyl, tetrazolyl, benzo[1,3]dioxolyl, 2,3-dihydro-benzo[1,4]dioxine, benzopyrimidyl, benzopyrazyl, benzofuranyl, indolyl, benzothienyl, benzoxazolyl, benzoisooxazolyl, benzothiazolyl, benzoisothiazolyl, quinolinyl, isoquinolinyl, benzimidazolyl, tetrahydroquinolinyl, and tetrahydroisoquinolinyl. Preferable aryl and heteroaryl groups include phenyl and pyridyl. The term “Ph” indicates a phenyl or a phenylene group, e.g., phenylene in —NR^{d}(CO)PhNR^{d}(CO)R^{b}, in R1 to R4.

The term “nonaromatic heterocycle” (e.g., the nonaromatic heterocyclic groups represented by NR^{c}₂ or NR^{j}₂) refers to non-aromatic ring systems typically having three to eight members, preferably five to six, in which one or more ring carbons, preferably one to four, are each replaced by a heteroatom such as N, O, or S. Examples of non-aromatic heterocyclic rings include 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothienyl, 3-tetrahydrothienyl, N-morpholinyl, 2-morpholinyl, 3-morpholinyl, 4-morpholinyl, N-thiomorpholinyl, 2-thiomorpholinyl, 3-thiomorpholinyl, 4-thiomorpholinyl, 1 -pyrrolidyl, 2-pyrrolidyl, 3-pyrorolidyl, 1-piperazyl, 2-piperazyl, 1-piperidyl, 2-piperidyl, 3-piperidyl, 4-piperidyl, 4-thiazolidyl, diazolonyl, N-substituted diazolonyl, 1-pthalimidyl, azetidyl, aziridyl, oxaziridyl, oxazolidyl, isooxazolidyl, thiazolidyl, isothiazolidyl, oxazinanyl, thiazinanyl, azepanyl, oxazepanyl, and thiazepanyl. Typically, the nonaromatic heterocycle groups represented by NR^{c}₂ and NR^{j}₂ are selected from optionally substituted pyrrolidyl, piperidyl, piperazyl, morpholinyl, and thiomorpholinyl., or preferably, unsubstituted piperidyl or morpholinyl.

The disclosed compounds can contain one or more chiral centers. For example, in structural formula I, the carbons in common between Rings A and C, and the carbon in Ring C between the nitrogen and Ring A can each be a chiral center. The presence of chiral centers in a molecule gives rise to stereoisomers. For example, a pair of optical isomers, referred to as “enantiomers”, exist for every chiral center in a molecule. A pair of diastereomers exist for every chiral center in a compound having two or more chiral centers. Where the structural formulas do not explicitly depict the stereochemistry of each chiral center, for example in structural formulas I-a to I-c, I-a′ to I-c′, I-a″, I-m, and the compounds in Table 1, it is to be understood that these formulas encompass enantiomers free from the corresponding optical isomer, racemic mixtures, mixtures enriched in one enantiomer relative to its corresponding optical isomer, a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s).

The term “alkyl” (e.g., the alkyl groups represented by R1 to R4, R^{a} to R^{d}, and R^{j}), used alone or as part of a larger moiety (e.g., aralkyl, alkoxy, alkylamino, alkylaminocarbonyl, haloalkyl), is a straight or branched non-aromatic hydrocarbon which is completely saturated. Typically, a straight or branched alkyl group has from 1 to about 10 carbon atoms, preferably from 1 to about 5 if not otherwise specified, Examples of suitable straight or branched alkyl group include methyl, ethyl, n-propyl, 2-propyl, n-butyl, sec-butyl, tert-butyl, pentyl, hexyl, heptyl or octyl. A C1 to C10 straight or branched alkyl group or a C3 to C8 cyclic alkyl group can also be referred to as a “lower alkyl” group. An “alkoxy” group refers to an alkyl group that is connected through an intervening oxygen atom, e.g., methoxy, ethoxy, 2-propyloxy, tert-butoxy, 2-butyloxy, 3-pentyloxy, and the like.

The terms “optionally halogenated alkyl”, and “optionally halogenated alkoxy”, as used herein, includes the respective group substituted with one or more of —F, —Cl, —Br, or —I.

The terms “alkanoyl”, “aroyl”, and the like, as used herein, indicates the respective group connected through an intervening carbonyl, for example, —(CO)CH₂CH₃, benzoyl, and the like. The terms “alkanoyloxy”, “aroyloxy”, and the like, as used herein, indicates the respective group connected through an intervening carboxylate, for example, —O(CO)CH₂CH₃, —O(CO)C₆H₅, and the like.

The term “cycloalkyl group” (e.g, the cycloalkyl groups represented by Ring A) is a cyclic alkyl group having from 3 to about 10 carbon atoms, preferably from 5 to 6. Examples of suitable cycloalkyl groups include cyclopropyl, cyclobutyl, cyclopentyl, cyclohexyl, cycloheptyl and cyclooctyl. A “cycloalkoxy” group refers to a cycloalkyl group that is connected through an intervening oxygen atom, e.g., cyclopentyloxy, cyclohexyloxy, and the like.

The term “cycloalkenyl” (e.g., the cycloalkyl groups represented by Ring A) includes nonaromatic cycloalkyl groups that contain one or more units of carbon-carbon unsaturation, i.e., carbon-carbon double bonds. A cycloalkenyl group includes, for example, cyclohexenyl or cyclopentenyl.

The terms “aralkyl”, “heteroaralkyl”, “cycloalkylalkyl”, and “nonaromatic heterocycloalkyl” refer to aryl, heteroaryl, cycloalkyl, and nonaromatic heterocycle groups, respectively, that are connected through an alkyl chain, e.g., benzyl, —CH₂H₂-pyridine, (3-cyclohexyl)propyl, and the like.

An “acyclic” group is a substituent that does not contain a ring. A “monocyclic” group contains only a single ring, for example, a phenyl ring that is not fused to another ring. A “polycyclic” group is a group that contains multiple fused rings, for example, naphthyl.

The term “derivative”, e.g., in the term “cycloalkyltetrahydroquinoline derivatives”, refers to compounds that have a common core structure, and are substituted with various groups as described herein. For example, all of the compounds represented in Table 1 are cycloalkyltetrahydroquinoline derivatives, and have structural formula I as a common core.

A line across a bond in a ring, for example, the line from HO₂C— in structural formulas I-b and I-c, indicates that the represented bond can be connected to any substitutable atom in the ring.

A “substitutable atom” is any atom such as nitrogen or carbon that can be substituted by replacing a hydrogen atom bound to the atom with a substituent. A “substitutable ring atom” in a ring, e.g., the substitutable ring carbons in Rings A to C, is any ring atom, e.g., a carbon or nitrogen, which can be substituted. For example, when X2 is a carbon, it can be bound to —H or substituted, e.g., with R2.

Suitable substituents are those that do not substantially interfere with the pharmaceutical activity of the disclosed compound. A compound or group can have one or more substituents, which can be identical or different. Examples of suitable substituents for a substitutable carbon atom in an alkyl, cycloalkyl, cycloalkenyl, non-aromatic heterocycle, aryl, or heteroaryl group include —OH, halogen (—Br, —Cl, —I and —F), —R, —OR, —CH₂R, —CH₂CH₂R, —OCH₂R, —CH₂OR, —CH₂CH₂OR, —CH₂OC(O)R, —O—COR, —COR, —SR, —SCH₂R, —CH₂SR, —SOR, —SO₂R, —CN, —NO₂, —COOH, —SO₃H, —NH_{2,} —NHR, —N(R)₂, —COOR, —CH₂COOR, —CH₂CH₂COOR, —CHO, —CONH₂, —CONHR, —CON(R)₂, —NHCOR, —NRCOR, —NHCONH₂, —NHCONRH, —NHCON(R)₂, —NRCONH₂, —NRCONRH, —NRCON(R)₂, —C(═NH)—NH₂, —C(═NH)—NHR, —C(═NH)—N(R)₂, —C(═NR)—NH₂, —C(═NR)—NHR, —C(═NR)—N(R)₂, —NH—C(═NH)—NH₂, —NH—C(═NR)—NHR, —NH—C(═NH)—N(R)₂, —NH—C(═NR)—NH₂, —NH—C(═NR)—NHR, —NH—C(═NR)—N(R)₂, —NRH—C(═NH)—NH₂, —NR—C(═NH)—NHR, —NR—C(═NH)—N(R)₂, —NR—C(═NR)—NH₂, —NR—C(═NR)—NHR, —NR—C(═NR)—N(R)₂, —SO₂NH₂, —SO₂NHR, —SO₂NR₂, —SH, —SOₖR (k is 0, 1 or 2) and —NH—C(═NH)—NH₂. Each R is independently an alkyl, cycloalkyl, benzyl, aromatic, heteroaromatic, or phenylamine group that is optionally substituted. Preferably, R is unsubstituted. In addition, —N(R)₂, taken together, can also form a substituted or unsubstituted heterocyclic group, (e.g., as for NR^{c}₂, and NR^{j}₂) such as pyrrolidinyl, piperidinyl, morpholinyl and thiomorpholinyl. Examples of substituents on group represented by R include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl.

Suitable substituents on the nitrogen of a heterocyclic group or heteroaromatic group include —R′, —N(R′)₂, —C(O)R′, —CO₂ R, —C(O)C(O)R′, —C(O)CH₂ C(O)R′, —SO₂R′, —SO₂ N(R′)₂, —C(═S)N(R′)₂, —C(═NH)—N(R′)₂, and —NR′ SO₂R′. R′ is hydrogen, an alkyl, alkoxy, cycloalkyl, cycloalkoxy, phenyl, phenoxy, benzyl, benzyloxy, heteroaromatic, or heterocyclic group that is optionally substituted. Examples of substituents on the groups represented by R′ include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl. Preferably, R′ is unsubstituted.

### EXEMPLIFICATION

### Example 1

### Synthesis of MurA Inhibitors of Structural Formula I-a

The disclosed compounds can be prepared by standard methods starting from appropriate commercially available starting materials.

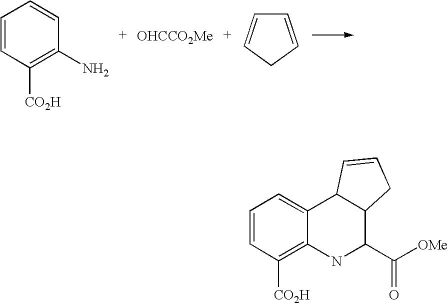

Concentrated HCl (1.7 mL, 20 mmol) was added to a solution of 2-aminobenzoic acid (2.74 g, 20 mmol) in 30 mL of methanol at 0°-5° C. After stirring for 15 min, glyoxylic acid methyl ester (2.8 M, 7.9 mL, 22 mmol) was. The mixture was stirred for 2 h at 0°-5° C., cyclopentadiene (1.6 mL, 20 mmol) was added. After stirring an additional 2 h at 0°-5° C., the solid product was collected by filtration and purified by silica gel column chromatography (petroleum ether-ethyl acetate, 2:1). The pure product was obtained as a white solid (2.6 g, 48% yield). See Ganem, B. 1989. Organizational Chemistry 2:127-128, the entire teachings of which are incorporated herein by reference.

Using the methods in the above example, compounds represented by structural formula I, i.e., Compounds II to LXXXIV and I-m (Table 1) were prepared by starting from appropriate reagents. In Table 1, structures depicting unfilled valences on N or O, i.e., are understood to be bonded to —H.

Compounds that are racemic, stereochemically enriched, or stereochemically pure can be prepared by an appropriate combination of methods selected from employing appropriate starting materials or reagents, crystallization, and chromatographic purification. See, for example, Ahuja, S. “Chiral Separations by Chromatography”, American Chemical Society, 2000; Ahuja, S. “Chiral Separations: Applications and Technology”, American Chemical Society, 1996, and references therein, the entire teachings of which are incorporated herein by reference.

### Example 2

### High Throughput Screen Identifies Likely MurA Inhibitors

A high throughput screen was employed on the compounds to identify the likely MurA inhibitors depicted in Table 1. The test conditions employed MurA and MurB (UDP-N-acetylmuramate:NADP+oxidoreductase, EC 1.1.1.158) coupled enzymatic reactions carried in 96-well reaction plates.

Using appropriate stock solutions, each well was prepared to contain a total volume of about 100 μL, containing 50 mM Tris-HCl (Tris(hydroxymethyl)aminomethane-HCl, pH 8.0), 20 mM KCl, 0.02% Brij®30 (Polyethylene glycol dodecyl ether), 0.5 mM DTT (dithiothreitol), 0.1 mM UDPAG (Uridine 5′-diphospho-N-acetylglucosamine), 0.1 mM phosphoenolpyruvate (PEP), 0.1 mM NADPH (nicotinamide adenine dinucleotide phosphate), 120 ng MurA, and 40 ng MurB. The preceding chemical reagents were obtained from Sigma, St. Louis Mo.; the enzymes were produced in house.

The wells were prepared without substrate (PEP and UDPAG) incubated for a half hour, combined with the substrate and each test compound, and the evidence of reaction was read after 1 hour of reaction time using a fluorescence spectrometer at 355/460 nM for 0. 1 second. Compounds that were associated with an increase in fluorescence over control solutions were identified as likely MurA inhibitors.

TABLE 1
MurA Inhibitors of Structural Formula I

| | | |
|---|---|---|
| 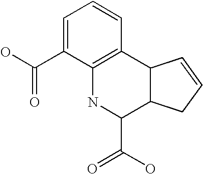 | 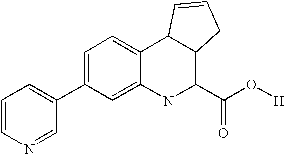 | 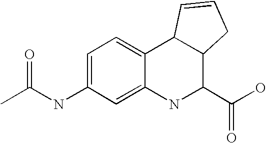 |
| II | V | IX |
| 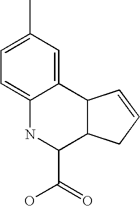 | 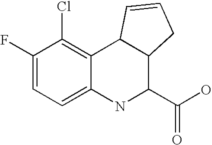 | 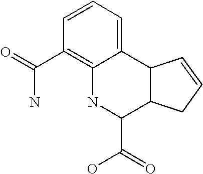 |
| III | VI | X |
| 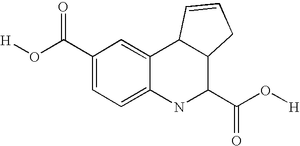 | 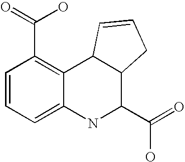 | 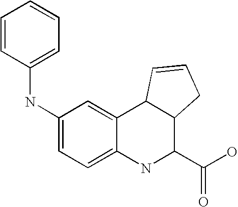 |
| IV | VII | XI |
| 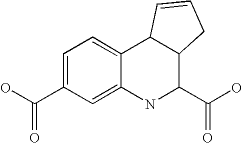 | 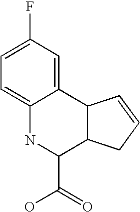 | 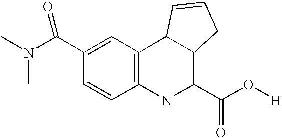 |
| I-m | VIII | XII |
| 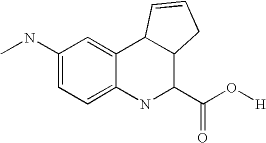 | 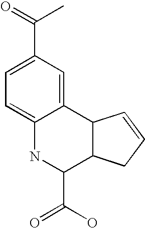 | 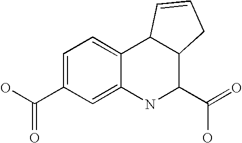 |
| XIII | XVII | XXI |
| 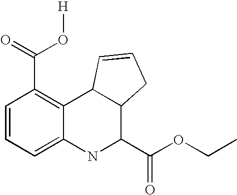 | 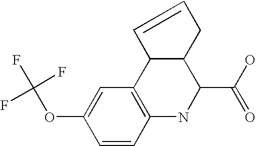 | 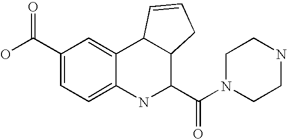 |
| XIV | XVIII | XXII |
| 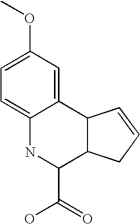 | 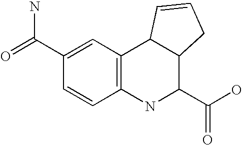 | 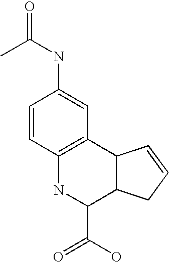 |
| XV | XIX | XXIII |
| 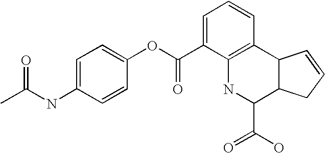 | 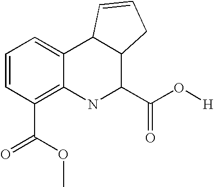 | 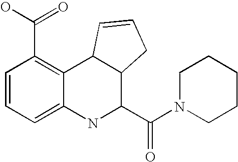 |
| XVI | XX | XXIV |
| 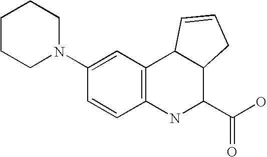 | 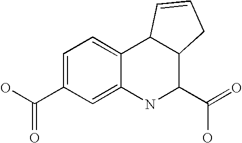 | 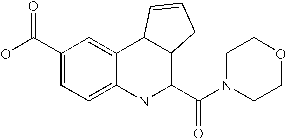 |
| XXV | XXX | XXXVII |
| 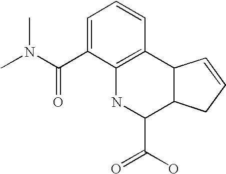 | 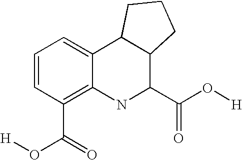 | 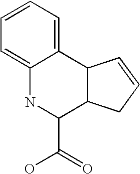 |
| XXVI | XXXI | XXXVIII |
| 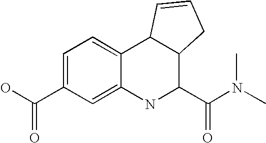 | 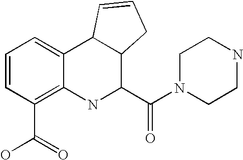 | 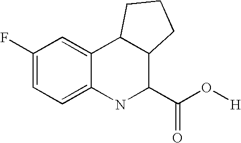 |
| XXVII | XXXV | XLVII |
| 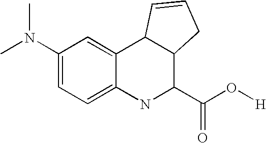 | 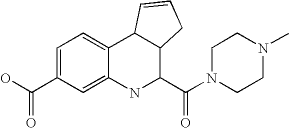 | 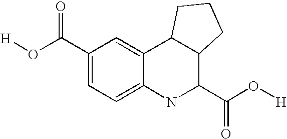 |
| XXVIII | XXXVI | XLIX |
| 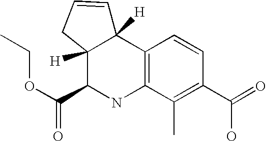 | 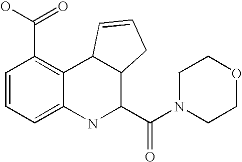 | 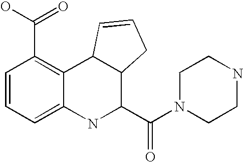 |
| LII | LX | LXXVI |
| 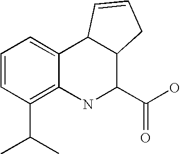 | 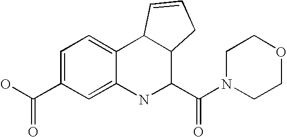 | |
| LVII | LXI | |
| 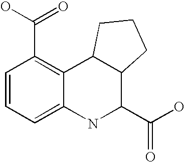 | 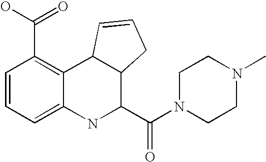 | |
| LVIII | LXII | |
| 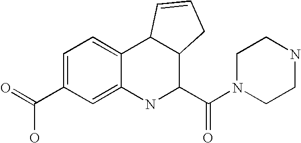 | 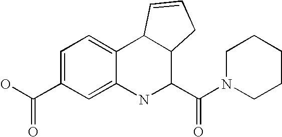 | |
| LIX | LXXV | |

### Example 3

### Kinetic Assay of Disclosed Inhibitors Shows Potent MurA Inhibition

A series of IC50 (inhibition Concentration at 50 percent) assays were performed in 96-well assay plates. About 60 μL of a buffer A1 was added into each well from column 1 to column 12. An additional 20 μL of buffer A1 was added into column 12. Buffer A1 was prepared to contain 50 mM HEPES pH 7.5(4-(2-hydroxyethyl)piperazine-1-ethanesulfonic acid), 20 mM KCl, 0.02% wt Brij 30, 0.001 mM UDPAG, 0.001 mM PEP, and 0.5 mM DTT.

Approximately 2 μL of compound solution was transferred by serial dilution from column 2 to column 11, resulting in a range of final compound concentrations from about 25 to about 0.049 μg/mL.

Approximately 20 μL of enzyme solution A2 was added into each well of column 1 through 11. Buffer A2 was prepared to contain 50 mM HEPES pH 7.5, 20 mM KCl, 0.02% wt Brij 30, 0.001 mM UDPAG, 0.001 mM PEP, 0.5 mM DTT, and 6 μg/mL MurA.

The plated solutions were incubated for half hour, after which approximately 20 μL of substrate solution B was added to each well, column 1 through 11, to initiate the reaction. Buffer B is prepared as 2 mM UDPAG, 0.4 mM PEP, 50 mM HEPES pH 7.5, 20 mM KCl, 0.02% wt Brij 30 and 0.5 mM DTT.

After reacting for 8 minutes, 150 μL of Malachite Green was added, the resulting combination incubated for 15 minutes at ambient temperature, and the reaction result was determined by measuring absorbance at 650 nm with a spectrometer.

The data were fit to a curve using Xlfit (ID Business Solutions, Cambridge, Mass.)). The IC₅₀ value was derived from the curve as the compound concentration that gave 50% inhibition of the enzymatic reaction. The results are depicted in Table 2.

TABLE 2
IC50 Inhibition Assay Reveals Potent MurA Inhibitors

| # | MURa IC50 |
|---|---|
| II | <5 |
| III | <5 |
| IV | <5 |
| I-m | <5 |
| V | <5 |
| VI | <5 |
| VII | <5 |
| VIII | <5 |
| IX | <5 |
| X | <5 |
| XI | <5 |
| XII | <5 |
| XIII | <5 |
| XIV | <5 |
| XV | <5 |
| XVI | <5 |
| XVII | <5 |
| XVIII | <5 |
| XIX | <5 |
| XX | <5 |
| XXI | <5 |
| XXII | ≧5, <33 |
| XXIII | ≧5, <33 |
| XXIV | ≧5, <33 |
| XXV | ≧5, <33 |
| XXVI | ≧5, <33 |
| XXVII | ≧5, <33 |
| XXVIII | ≧5, <33 |
| XXX | ≧33 |
| XXXI | ≧33 |
| XXXV | ≧33 |
| XXXVI | ≧33 |
| XXXVII | ≧33 |
| XXXVIII | ≧33 |
| XLVII | ≧33 |
| XLIX | ≧33 |
| LII | ≧33 |
| LVII | ≧33 |
| LVIII | ≧33 |
| LIX | ≧33 |
| LX | ≧33 |
| LXI | ≧33 |
| LXII | ≧33 |
| LXXV | ≧33 |
| LXXVI | ≧33 |

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a subject for a bacterial infection, comprising administering to a subject in need of treatment for a bacterial infection an effective amount of a compound represented by structural formula I: 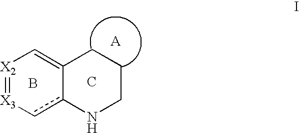
or a pharmaceutically acceptable salt, solvate, or hydrate thereof, wherein:
Ring A is a 5 or 6 membered cycloalkyl or cycloalkenyl group, optionally substituted with halogen or optionally′halogenated C1-C3 alkyl or alkoxy;
X2 and X3 are each carbon, or one is nitrogen and the other is carbon; and
Rings B and C are optionally and independently substituted at any substitutable ring carbon, provided that one or two substitutable ring carbons in Rings B and C are substituted with an acidic group.

2 .The method of claim 1, wherein the subject is a human.

3. The method of claim 2, wherein the infection is caused by a bacterium that expresses phosphoenolpyruvate: UDP-N-acetyl-D-glucosamine 1-carboxyvinyltransferase.

4. The method of claim 2, wherein the infection is caused by a bacterium of a genus selected Allochromatium, Acinetobacter, Bacillus, Campylobacter, Chlamydia, Chlamydophila Clostridium, Citrobacter, Escherichia, Enterobacter, Enterococcus, Francisella, Haemophilus, Helicobacter, Klebsiella, Listeria, Moraxella, Mycobacterim, Neisseria, Proteus, Pseudomonas, Salmonella, Serratia, Shigella, Stenotrophomonas, Staphyloccocus, Streptococcus, Synechococcus, Vibrio, and Yersina.

5. The method of claim 4 wherein the bacterial infection is from [correct list?] Allochromatium vinosum, Acinetobacter baumanii, Bacillus anthracis, Campylobacter jejuni Chlamydia trachomatis, Chlamydia pneumoniae, Clostridium spp., Citrobacter spp., Escherichia coli, Enterobacter spp., Enterococcus faecalis., Enterococcus faecium, Francisella tularensis, Haemophilus influenzae, Helicobacter pylori, Klebsiella spp., Listeria monocytogenes, Moraxella catarrhalis, Mycobacterium tuberculosis, Neisseria meningitidis, Neisseria gonorrhoeae, Proteus mirabilis, Proteus vulgaris, Pseudomonas aeruginosa, Salmonella spp., Serratia spp., Shigella spp., Stenotrophomonas maltophilia, Staphyloccocus aureus, Staphyloccocus epidermidis, Streptococcus pneumoniae, Streptococcus pyogenes, Streptococcus agalactiae, Yersina pestis, and Yersina enterocolitica.

6. The method of claim 5 wherein the acidic group is selected from —(CO)OH, —(CS)OH, —(SO)OH, —SO₃H, —OSO₃H, —P(OR^{a})(OH), —(PO)(OR^{a})(OH), —O(PO)(OR^{a})(OH), or —B(OR^{a})(OH), wherein R^{a} is —H or optionally substituted aryl, aralkyl, heteroaryl, heteroaralkyl, or C1 to C4 alkyl.

7. The method of claim 6, wherein the compound is represented by structural formula I-a:

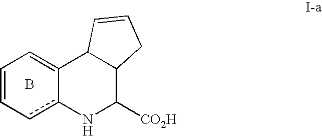

8. The method of claim 7, wherein the compound is represented by structural formula I-a′: 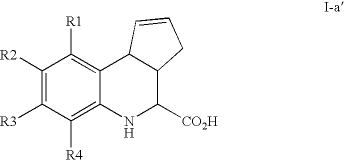
wherein:
R1, R2, R3, and R4 are independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —(CO)NR^{c}₂, —NR^{c}_{2,} —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, nonaromatic heterocycle, C1 to C4 alkyl, C1 to C4 alkoxy, C1 to C4 hydroxy alkyl, or C2 to C6 alkoxyalkyl; wherein:
each R^{b} and R^{d} is independently —H or optionally substituted aryl, aralkyl, heteroaryl, heteroaralkyl, or C1 to C4 alkyl; and
each R^{c} is independently —H or optionally substituted C1 to C4 alkyl, aryl, or aralkyl, or NR^{c}₂ is an optionally substituted nonaromatic heterocycle.

9. The method of claim 8 wherein at least two of R1 to R4 are —H.

10. The method of claim 9 wherein:
one or two of R1 to R4 are each independently —F, —Cl, —Br, —(CO)R^{b}, —(CO)OR^{b}, —(CO)NR^{c}₂, —NR^{c}₂, —NR^{d}(CO)R^{b}, —NR^{d}(CO)OR^{b}, —NR^{d}(CO)NR^{c}₂, —NR^{d}(CO)PhNR^{d}(CO)R^{b}, or optionally substituted phenyl, benzyl, pyridyl, methylpyridyl, or optionally halogenated C1 to C4 alkyl or C1 to C4 alkoxy;
wherein each R^{b}, R^{c}, and R^{d} is independently —H, or optionally substituted C1 to C4 alkyl or phenyl, or each NR^{c}₂ is an optionally substituted morpholinyl, piperidyl, or piperazyl.

11. The method of claim 10 wherein the compound is represented by one of the following structural formulas:

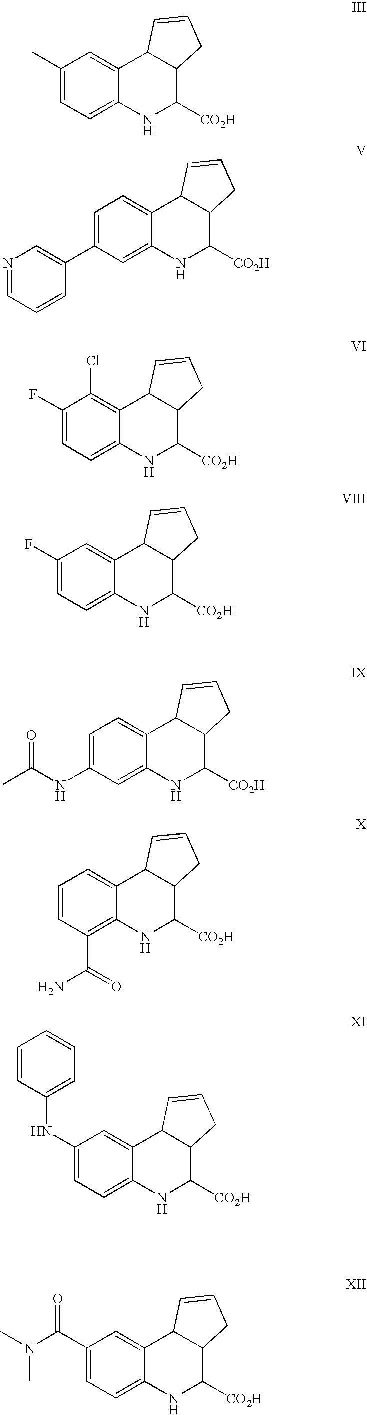

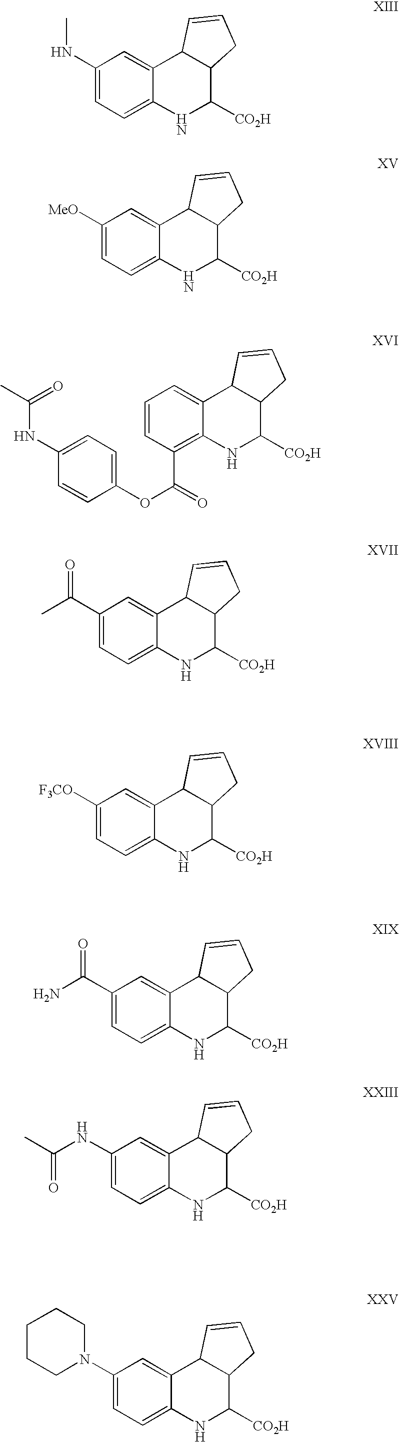

12. The method of claim 8 wherein at least one of R1 to R4 is —CO₂H, or a C1 to C4 alkyl ester thereof.

13. The method of claim 12 wherein the compound is represented by one of the following structural formulas:

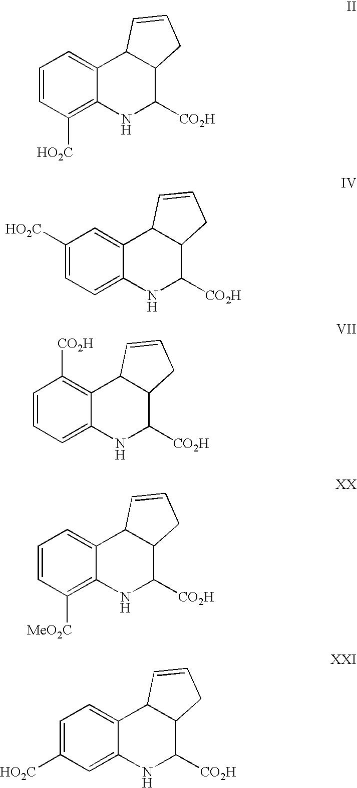

14. The method of claim 6, wherein the compound is represented by structural formula I-b: 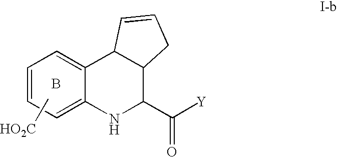
wherein Y is optionally substituted C1 to C4 alkyl, C1 to C4 alkoxy, phenyl, pyridyl, or —NR^{j}_{2,} wherein each R^{j} is independently —H, C1 to C4 alkyl, aryl, or aralkyl, or NR^{j}₂ is a nonaromatic heterocycle.

15. The method of claim 14, wherein the compound is represented by structural formula I-b′: 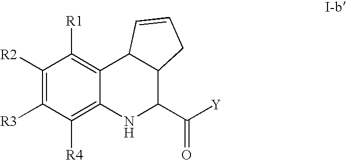
wherein:
R1, R2, R3, and R4 are independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, nonaromatic heterocycle, C1 to C4 alkyl, C1 to C4 alkoxy, C1 to C4 hydroxy alkyl, or C2 to C6 alkoxyalkyl, wherein at least one of R1 to R4 is —CO₂H;
wherein:
each R^{b} and R^{d} is independently —H or optionally substituted aryl, aralkyl, heteroaryl, heteroaralkyl, or C1 to C4 alkyl; and
each R^{c} is independently —H or optionally substituted C1 to C4 alkyl, aryl, or aralkyl, or NR^{c}₂ is an optionally substituted nonaromatic heterocycle.

16. The method of claim 15 wherein at least two of R1 to R4 are —H.

17. The method of claim 16, wherein the compound is represented by one of the following structural formulas:

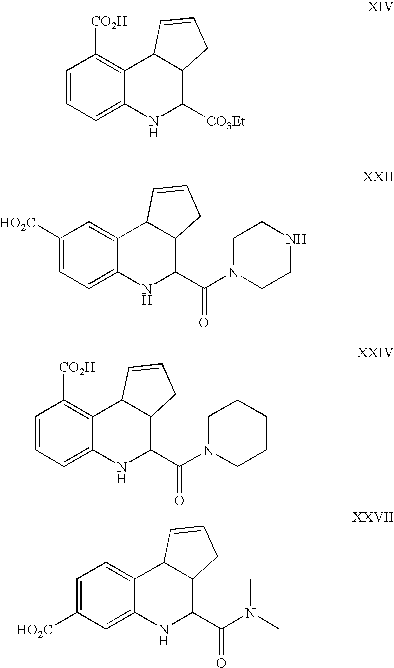

18. The method of claim 6, wherein the compound is represented by structural formula I-c:

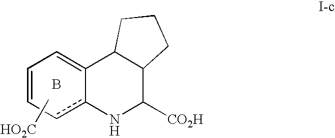

19. The method of claim 18, wherein the compound is represented by structural formula I-c′: 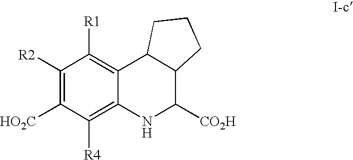
wherein:
R1, R2, and R4 are independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, nonaromatic heterocycle, C1 to C4 alklyl, C1 to C4 alkoxy, C1 to C4 hydroxy alkyl, or C2 to C6 alkoxyalkyl;
wherein:
each R^{b} and R^{d} is independently —H or optionally substituted aryl, aralkyl, heteroaryl, heteroaralkyl, or C1 to C4 alkyl; and
each R^{c} is independently —H or optionally substituted C1 to C4 alkyl, aryl, or aralkyl, or NR^{c}₂ is an optionally substituted nonaromatic heterocycle.

20. The method of claim 19, wherein R1, R2, and R4 are independently —H, —F, —Cl, —Br, —NO₂, —CN, —(CO)R^{b}, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or optionally halogenated C1 to C4 hydroxy alkyl, C1 to C4 alkyl, or C1 to C4 alkoxy;
wherein
each R^{b}, R^{c} and R^{d} is independently —H or C1 to C4 alkyl; or NR^{c}₂ is a nonaromatic heterocycle.

21. The method of claim 20 wherein at least two of R1, R2, and R4 are —H.

22. The method of claim 21 wherein the compound is represented by structural formula I-m:

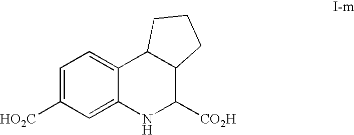

23. A compound represented by structural formula I-a′: 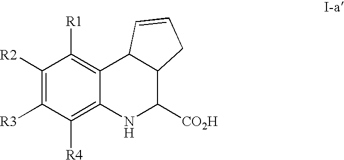
or a pharmaceutically acceptable salt, solvate, or hydrate thereof, wherein:
R1, R2, R3, and R4 are independently —H, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —NR^{c}₂, —NR^{d}(CO)R^{b}, NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, or nonaromatic heterocycle;
wherein:
each R^{b} and R^{d} is independently —H or optionally substituted aryl, aralkyl, heteroaryl, heteroaralkyl, or C1 to C4 alkyl; and
each R^{c} is independently —H or optionally substituted C1 to C4 alkyl, aryl, or aralkyl, or NR^{c}₂ is an optionally substituted nonaromatic heterocycle.

24. The compound of claim 23 wherein at least two of R1 to R4 are —H.

25. The compound of claim 24 wherein:
one or two of R1 to R4 are each independently —(CO)R^{b}, —(CO)OR^{b}, —(CO)NR^{c}₂, —NR^{c}₂, —NR^{d}(CO)R^{b}, —NR^{d}(CO)OR^{b}, —NR^{d}(CO)NR^{c}₂, —NR^{d}(CO)PhNR^{d}(CO)R^{b}, or optionally substituted phenyl, benzyl, pyridyl, or methylpyridyl;
wherein each R^{b}, R^{c}, and R^{d} is independently —H, or optionally substituted C1 to C4 alkyl or phenyl, or each NR^{c}₂ is an optionally substituted morpholinyl, piperidyl, or piperazyl.

26. The compound of claim 25 wherein the compound is represented by one of the following structural formulas:

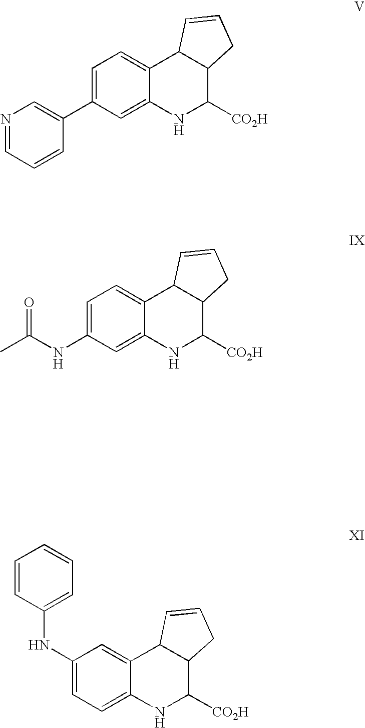

27. A compound represented by structural formula I-a″: 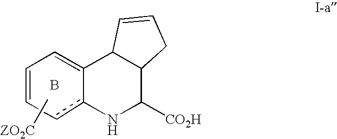
or a pharmaceutically acceptable salt, solvate, or hydrate thereof, wherein Ring B is optionally substituted at any substitutable ring carbon, and Z is —H or a C1 to C4 alkyl group.

28. The compound of claim 27, wherein the compound is represented by structural formula I-a′: 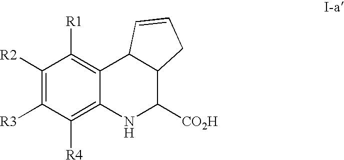
wherein:
R1, R2, R3, and R4 are independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂ , —O(PO)(OR^{b})₂, —B(OR^{b})₂, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, nonaromatic heterocycle, C1 to C4 alkyl, C1 to C4 alkoxy, C1 to C4 hydroxy alkyl, or C2 to C6 alkoxyalkyl, wherein at least one of R1 to R4 is —(CO)OR^{b};
wherein:
each R^{b} and R^{d} is independently —H or optionally substituted aryl, aralkyl, heteroaryl, heteroaralkyl, or C1 to C4 alkyl; and
each R^{c} is independently —H or optionally substituted C1 to C4 alkyl, aryl, or aralkyl, or NR^{c}₂ is an optionally substituted nonaromatic heterocycle.

29. The compound of claim 28, wherein the compound is represented by one of the following structural formulas:

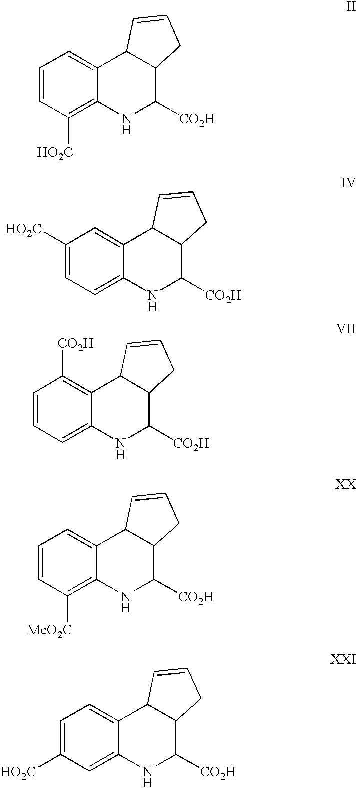

30. A compound represented by structural formula I-b: 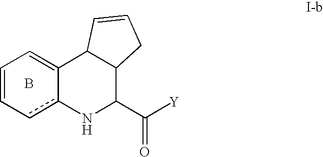
or a pharmaceutically acceptable salt, solvate, or hydrate thereof, wherein:
Ring B is optionally substituted at any substitutable ring carbon, provided that one or two substitutable ring carbons in Ring B are substituted with an acidic group; and
Y is optionally substituted C1 to C4 alkyl, C1 to C4 alkoxy, phenyl, pyridyl, or —NR^{j}₂;
wherein each R^{j} is independently —H, C1 to C4 alkyl, aryl, or aralkyl, or NR^{j}₂ is a nonaromatic heterocycle.

31. The compound of claim 30 wherein the acidic group is selected from —(CO)OH, —(CS)OH, —(SO)OH, —SO₃H, —OSO₃H, —P(OR^{a})(OH), —(PO)(OR^{a})(OH), —O(PO)(OR^{a})(OH), or —B(OR^{a})(OH), wherein R^{a} is —H or optionally substituted aryl, aralkyl, heteroaryl, heteroaralkyl, or C1 to C4 alkyl.

32. The compound of claim 31, wherein the compound is represented by structural formula I-b′: 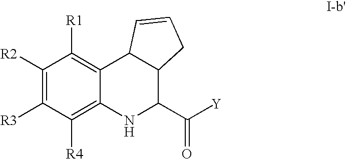
wherein:
R1, R2, R3, and R4 are independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —(CO)NR^{c}₂, —NR^{c}₂, NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, nonaromatic heterocycle, C1 to C4 alkyl, C1 to C4 alkoxy, C1 to C4 hydroxy alkyl, or C2 to C6 alkoxyalkyl; provided that at least one of R1 to R4 is —CO₂H;
wherein
each R^{b} and R^{d} is independently —H or optionally substituted aryl, aralkyl, heteroaryl, heteroaralkyl, or C1 to C4 alkyl; and
each R^{c} is independently —H or optionally substituted C1 to C4 alkyl, aryl, or aralkyl, or NR^{c}₂ is an optionally substituted nonaromatic heterocycle.

33. The compound of claim 32 wherein at least two of R1 to R4 are —H.

34. The compound of claim 33 wherein one of R1 to R4 is —CO₂H.

35. The compound of claim 34, wherein the compound is represented by one of the following structural formulas:

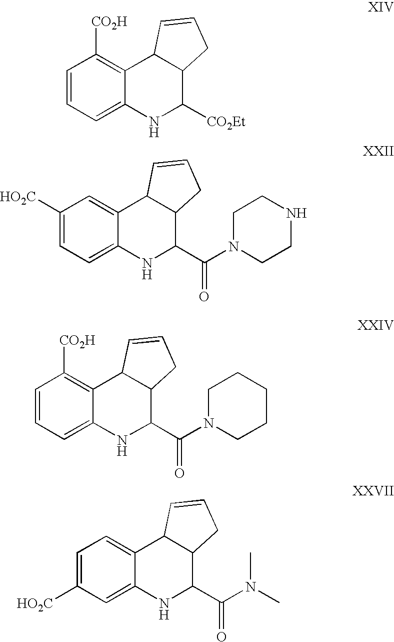

36. A compound represented by structural formula I-c: 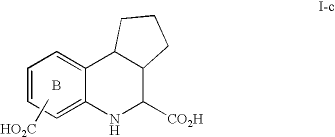
or a pharmaceutically acceptable salt, solvate, or hydrate thereof, wherein Ring B is optionally substituted at any substitutable ring carbon.

37. The compound of claim 36, wherein the compound is represented by structural formula I-c′: 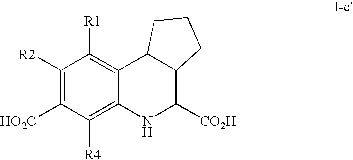
wherein:
R1, R2, and R4 are independently —H, halogen, —NO₂, —CN, —(CO)R^{b}, —(CO)OR^{b}, —(CO)O(CO)R^{b}, —(CS)OR^{b}, —(CS)R^{b}, —(SO)OR^{b}, —SO₃R^{b}, —OSO₃R^{b}, —P(OR^{b})₂, —(PO)(OR^{b})₂, —O(PO)(OR^{b})₂, —B(OR^{b})₂, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or an optionally substituted aryl, aralkyl, heteroaryl, heteroaralkyl, C3 to C7 cycloalkyl, nonaromatic heterocycle, C1 to C4 alkyl, C1 to C4 alkoxy, C1 to C4 hydroxy alkyl, or C2 to C6 alkoxyalkyl;
wherein:
each R^{b} and R^{d} is independently —H or optionally substituted aryl, aralkyl, heteroaryl, heteroaralkyl, or C1 to C4 alkyl; and
each R^{c} is independently —H or optionally substituted C1 to C4 alkyl, aryl, or aralkyl, or NR^{c}₂ is an optionally substituted nonaromatic heterocycle.

38. The compound of claim 37, wherein R1, R2, and R4 are independently —H, —F, —Cl, —Br, —NO₂, —CN, —(CO)R^{b}, —(CO)NR^{c}₂, —NR^{c}₂, —NR^{d}(CO)R^{b}, —NR^{d}(CO)OR^{b}, —NR^{d}(CO)NR^{c}₂, —SO₂NR^{c}₂, —NR^{d}SO₂R^{b}, or optionally halogenated C1 to C4 hydroxy alkyl, C1 to C4 alkyl, or C1 to C4 alkoxy;
wherein each R^{b}, R^{c} and R^{d} is independently —H or C1 to C4 alkyl; or NR^{c}₂ is a nonaromatic heterocycle.

39. The compound of claim 38 wherein two of R1, R2, and R4 are —H.

40. The compound of claim 39 wherein the compound is represented by structural formula I-m:

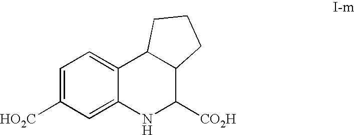

41. A method of identifying a MurA inhibitor, comprising:
contacting MurA with phosphoenolpyruvate and a test compound;
determining a reaction rate between the phosphoenolpyruvate and MurA; and
identifying the test compound as a MurA inhibitor when the rate of reaction in the presence of the test compound is less than a reaction rate in the absence of the test compound.

42. The method of claim 41, further comprising conducting the reaction in the presence of MurB and uridine 5′-diphospho-N-acetylglucosamine.
